(19) United States
(12) Patent Application Publication
(10) Pub. No.: US 2012/0045414 A1
(43) Pub. Date: Feb. 23, 2012

(54) ADJUVANT COMBINATIONS COMPRISING A MICROBIAL TLR AGONIST, A CD40 OR 4-1BB AGONIST, AND OPTIONALLY AN ANTIGEN AND THE USE THEREOF FOR INDUCING A SYNERGISTIC ENHANCEMENT IN CELLULAR IMMUNITY

(76) Inventor: Dave Delucia, Norwich, VT (US)

(21) Appl. No.: 13/164,853

(22) Filed: Jun. 21, 2011

Related U.S. Application Data
(63) Continuation of application No. 11/931,237, filed on Oct. 31, 2007, now abandoned.
(60) Provisional application No. 60/863,695, filed on Oct. 31, 2006.

(51) Int. Cl. A61K 38/21; A61P 35/00; A61P 31/12; A61P 31/10; A61P 37/00; A61P 31/04

(52) U.S. Cl. 424/85.4

(57) ABSTRACT

Adjuvant combinations comprising at least one microbial TLR agonist such as a whole virus, bacterium or yeast or portion thereof such a membrane, spheroplast, cytoplast, or ghost, a CD40 or 4-1BB agonist and optionally an antigen wherein all 3 moieties may be separate or comprise the same recombinant microorganism or virus are disclosed. The use of these immune adjuvants for treatment of various chronic diseases such as cancers and HIV infection is also provided.

### PRIORITY INFORMATION

This application is a new Continuation of U.S. application Ser. No. 11/931,237 filed on Oct. 31, 2007, which claims benefit of priority to provisional application Ser. No. 60/863,695 filed on Oct. 31, 2006, these applications are incorporated by reference in their entirety herein.

### FIELD OF THE INVENTION

The invention generally relates to synergistic adjuvant combinations which promote antigen specific cellular immunity. The use of these immune adjuvants for treating various chronic diseases including cancer, infectious diseases, autoimmune diseases, allergic and inflammatory diseases is also taught.

### BACKGROUND OF THE INVENTION

The body's defense system against microbes as well as the body's defense against other chronic diseases such as those affecting cell proliferation is mediated by early reactions of the innate immune system and by later responses of the adaptive immune system. Innate immunity involves mechanisms that recognize structures which are for example characteristic of the microbial pathogens and that are not present on mammalian cells. Examples of such structures include bacterial liposaccharides, (LPS) viral double stranded DNA, and unmethylated CpG DNA nucleotides. The effector cells of the innate immune response system comprise neutrophils, macrophages, and natural killer cells (NK cells). In addition to innate immunity, vertebrates, including mammals, have evolved immunological defense systems that are stimulated by exposure to infectious agents and that increase in magnitude and effectiveness with each successive exposure to a particular antigen. Due to its capacity to adapt to a specific infection or antigenic insult, this immune defense mechanism has been described as adaptive immunity. There are two types of adaptive immune responses, called humoral immunity, involving antibodies produced by B lymphocytes, and cell-mediated immunity, mediated by T lymphocytes.

Two types of major T lymphocytes have been described, CD8+ cytotoxic lymphocytes (CTLs) and CD4 helper cells (Th cells). CD8+ T cells are effector cells that, via the T cell receptor (TCR), recognize foreign antigens presented by class I MEW molecules on, for instance, virally or bacterially infected cells. Upon recognition of foreign antigens, CD8+ cells undergo an activation, maturation and proliferation process. This differentiation process results in CTL clones which have the capacity of destroying the target cells displaying foreign antigens. T helper cells on the other hand are involved in both humoral and cell-mediated forms of effector immune responses. With respect to the humoral, or antibody immune response, antibodies are produced by B lymphocytes through interactions with Th cells. Specifically, extracellular antigens, such as circulating microbes, are taken up by specialized antigen-presenting cells (APCs), processed, and presented in association with class II major histocompatibility complex (MEW) molecules to CD4+ Th cells. These Th cells in turn activate B lymphocytes, resulting in antibody production. The cell-mediated, or cellular, immune response, in contrast, functions to neutralize microbes which inhabit intracellular locations, such as after successful infection of a target cell. Foreign antigens, such as for example, microbial antigens, are synthesized within infected cells and resented on the surfaces of such cells in association with Class I MI-IC molecules. Presentation of such epitopes leads to the above-described stimulation of CD8+ CTLs, a process which in turn is also stimulated by CD4+ Th cells. Th cells are composed of at least two distinct subpopulations, termed Th1 and Th2 cells. The Th1 and Th2 subtypes represent polarized populations of Th cells which differentiate from common precursors after exposure to antigen.

Each T helper cell subtype secretes cytokines that promote distinct immunological effects that are opposed to one another and that cross-regulate each other's expansion and function. Th1 cells secrete high amounts of cytokines such as interferon (IFN) gamma, tumor necrosis factor-alpha (TNF-alpha), interleukin-2 (IL-2), and IL-12, and low amounts of IL-4. Th1 associated cytokines promote CD8+ cytotoxic T lymphocyte T lymphocyte (CTL) activity and are most frequently associated with cell-mediated immune responses against intracellular pathogens. In contrast, Th2 cells secrete high amounts of cytokines such as IL-4, IL-13, and IL-10, but low IFN-gamma, and promote antibody responses. Th2 responses are particularly relevant for humoral responses, such as protection from anthrax and for the elimination of helminthic infections.

Whether a resulting immune response is Th1 or Th2-driven largely depends on the pathogen involved and on factors in the cellular environment, such as cytokines. Failure to activate a T helper response, or the correct T helper subset, can result not only in the inability to mount a sufficient response to combat a particular pathogen, but also in the generation of poor immunity against reinfection. Many infectious agents are intracellular pathogens in which cell-mediated responses, as exemplified by Th1 immunity, would be expected to play an important role in protection and/or therapy. Moreover, for many of these infections it has been shown that the induction of inappropriate Th2 responses negatively affects disease outcome. Examples include M tuberculosis, S. mansoni, and also counterproductive Th2-like dominated immune responses. Lepromatous leprosy also appears to feature a prevalent, but inappropriate, Th2-like response. HIV infection represents another example. There, it has been suggested that a drop in the ratio of Th1-like cells to other Th cell populations can play a critical role in the progression toward disease symptoms.

As a protective measure against infectious agents, vaccination protocols for protection from some microbes have been developed. Vaccination protocols against infectious pathogens are often hampered by poor vaccine immunogenicity, an inappropriate type of response (antibody versus cell-mediated immunity), a lack of ability to elicit long-term immunological memory, and/or failure to generate immunity against different serotypes of a given pathogen. Current vaccination strategies target the elicitation of antibodies specific for a given serotype and for many common pathogens, for example, viral serotypes or pathogens. Efforts must be made on a recurring basis to monitor which serotypes are prevalent around the world. An example of this is the annual monitoring of emerging influenza A serotypes that are anticipated to be the major infectious strains.

To support vaccination protocols, adjuvants that would support the generation of immune responses against specific infectious diseases further have been developed. For example, aluminum salts have been used as a relatively safe and effective vaccine adjuvants to enhance antibody responses to certain pathogens. One of the disadvantages of such adjuvants is that they are relatively ineffective at stimulating a cell-mediated immune response and produce an immune response that is largely Th2 biased.

It is now widely recognized that the generation of protective immunity depends not only on exposure to antigen, but also the context in which the antigen is encountered. Numerous examples exist in which introduction of a novel antigen into a host in a non-inflammatory context generates immunological tolerance rather than long-term immunity whereas exposure to antigen in the presence of an inflammatory agent (adjuvant) induces immunity. (Mondino et al., Proc. Natl. Acad. Sci., USA 93:2245 (1996); Pulendran et al., J. Exp. Med. 188:2075 (1998); Jenkins et al., Immunity 1:443 (1994); and Kearney et al., Immunity 1:327 (1994)). Since it can mean the difference between tolerance and immunity, much effort has gone into discovering the “adjuvants” present within infectious agents that stimulate the molecular pathways involved in creating the appropriate immunogenic context of antigen presentation. It is now known that a good deal of the adjuvant activity is due to interactions of microbial and viral products with different members of the Toll Like Receptors (TLRs) expressed on immune cells (Beutler et al, Mol. Immunol. 40:845 (2004); Kaisho B., Biochim. Biophys. Acta, 1589 (2002):1; Akira et al., Scand. J Infect. Dis. 35:555 (2003); and Takeda K. and Akira S Semin. Immunol. 16:3 (2004)). The TLRs are named for their homology to a molecule in the Drosophila, called Toll, which functions in the development thereof and is involved in anti-microbial immunity (Lemaitre et al., Cell 86:973 (1996); and Hashimoto et al., Cell 52:269 (1988)).

Early work showed the mammalian homologues to Toll and Toll pathway molecules were critical to the ability of cells of the innate immune system to respond to microbial challenges and microbial byproducts (Medzhitov et al., Nature 388:394 (1997); Medzhitov et al., Mol. Cell 2:253 (1998); Medzhitov et al., Semin. Immunol. 10:351 (2000); Medzhitov et al., Trends Microbiol. 8:452 (2000); and Janeway et al., Annu Rev. Immunol. 20:197 (2002)). Since the identification of LPS as a TLR4 agonist (Poltorok et al., Science 282:2085 (1998)) numerous other TLR agonists have been described such as tri-acyl lipopeptides (TLR1), peptidoglycan, lipoteichoic acid and Pam3cys (TLR2), dsRNA (TLR3), flagellin (TLR5), diacyl lipopeptides such as Ma1p-2 (TLR6), imidazoquinolines and single stranded RNA (TLR7,8), bacterial DNA, unmethylated CpG DNA sequences, and even human genomic DNA antibody complexes (TLR9). Takeuchi et al. Int Immunol 13:933 (2001); Edwards et al., J Immunol 169:3652 (2002); Hayashi et al., Blood, 102:2660 (2003); Nagase et al., J Immunol. 171:3977 (2003).

As noted above flagellin in particular has been previously identified as a TLR5 agonist. Based on this property the use thereof as an immune potentiator has been suggested by some groups. For example Medzhitov et al., US 20050163764 published Jul. 28, 2005 suggest the use of flagellin and other TLR agonists for treating gastrointestinal injury in a mammal by oral or mucosal administration. Also, Aderem et al., US 20050147627 published Jul. 7, 2005 teach flagellin peptides that function as TLR5 agonists and use thereof to enhance antigen-specific immune responses by co-administration of the flagellin peptide and the antigen. Further, Aderem et al. US 2003004429 published Mar. 6, 2003 teach purported flagellin peptides that function as TLR5 agonists and the use thereof to treat conditions selected from proliferative diseases (cancer) autoimmune diseases, infectious diseases and inflammatory diseases. They further disclose that this administration may be combined with an immunomodulatory molecule which may be fused thereto and may comprise an antibody, cytokine or growth factor. Still further, Dow et al., US 20050013812 published Jan. 20, 2005 teach purported vaccines comprising a toll receptor ligand and a delivery vehicle for use in treating various diseases including cancers, infectious diseases, allergic diseases, autoimmune diseases and autoimmune diseases.

The involvement of TLRs in immunity is at least 2-fold, first as direct activators of the innate immune system, such as DCs, monocytes, macrophages, NK cells, esinophils, and neutrophils (17-20) to induce a cascade of cytokines and chemokines like IFNalpha, IL-12, IL-6, IL-8, MIPlalpha and beta, and MCP-1. (Medzhitov et al., Trends Microbiol. 8:452 (2002); Kaisho et al., Cur. Mol. Med. 3:759 (2003); Kopp and Medzhitov Curr Opin. Immunol. 15:396 (2003) and Beutler et al., J Leukoc Biol. 74:479 (2003)). DCs stimulated by various TLRs become activated to increase surface expression of costimulatory markers and migrate from the tissues and marginal zones into the T cell rich area of lymphoid tissues (De Smedt et al., J Exp Med 184:1413 (1996); Doxsee et al., J Immunol 171:1156 (2003); Reis e Sousa et al., J Exp Med 186:1819 (1997); and Suzuki et al., Dermatology 114:135 (2000)). These activated DCs are ideal for the presentation of antigens, gleaned from the peripheral tissues and circulation, to CD4 and CD8+ T cells within the T cell zones. Thus, TLR stimulation induces immediate innate effector functions and also creates the necessary conditions for the initiation of adaptive immunity.

TLR agonists alone are poor adjuvants for eliciting cellular immunity. Given their ability to mediate DC activation, cytokine production, costimulatory marker expression, and migration into T cell areas of lymphoid tissue, TLR agonists would seem to be optimal for use as vaccine adjuvants. However, when compared to an actual infection, the use of purified TLR agonists as vaccine adjuvants has been disappointing at best, at least with respect to the generation of T responses. Within 6-9 days after infection with many viruses and bacteria, either in animal models or in the clinic, the infected host often is capable of generating pathogen-specific T cell responses constituting 20-50% of the total circulating CD8+ T cells (Busch et al., Immunol Lett 65:93 ((1999); Busch et al., J Exp Med. 189:701 (1999); Butz et al., Adv Exp Med Biol 452:111 (1998); Butz et al., Immunity 8:167 (1998)). By contrast, the generation of detectable T cell responses using only an antigen and a TLR agonist(s) often requires multiple immunizations and even then the magnitude of the T cell response is rarely better than 5-10% of the circulating CD8+ T cells (Tritel et al., J Imunol 171:2539 (2003); Will-Reece et al., J Immunol 174:7676 (2005); Rhee et al., J Exp Med 195:1565 (2002); Lore et al., J Immunol 171:4320 (2003); Ahonen et al., J Exp Med 199:775 (2004)). Thus the reduction of an infectious agent down to its antigens and TLR agonists does not reconstitute the magnitude of cellular immunity generated by the actual infection.

Another molecule known to regulate adaptive immunity is CD40. CD40 is a member of the TNF receptor superfamily and is essential for a spectrum of cell-mediated immune responses and required for the development of T cell dependent humoral immunity (Aruffo et al., Cell 72:291 (1993); Farrington et al., Proc Natl Acad Sci., USA 91:1099 (1994); Renshaw et al., J Exp Med 180:1889 (1994)). In its natural role, CD40-ligand expressed on CD4+ T cells interacts with CD40 expressed on DCs or B cells, promoting increased activation of the APC and, concomitantly, further activation of the T cell (Liu et al Semin Immuno! 9:235 (1994); Bishop et al., Cytokine Growth Factor Rev 14:297 (2003)). For DCs, CD40 ligation classically leads to a response similar to stimulation through TLRs such as activation marker upregulation and inflammatory cytokine production(Quezada et al. Annu Rev Immunol 22:307 (2004); O'Sullivan B and Thomas R Crit Rev Immunol 22:83 (2003)) Its importance in CD8 responses was demonstrated by studies showing that stimulation of APCs through CD40 rescued CD4-dependent CD8+ T cell responses in the absence of CD4 cells (Lefrancois et al., J Immunol. 164:725 (2000); Bennett et al., Nature 393:478 (1998); Ridge et al., Nature 393:474 (1998); Schoenberger et al., Nature 393:474 (1998). This finding sparked much speculation that CD40 agonists alone could potentially rescue failing CD8+ T cell responses in some disease settings.

Other studies, however, have demonstrated that CD40 stimulation alone insufficiently promotes long-term immunity. In some model systems, anti-CD40 treatment alone insufficiently promoted long-term immunity. In some model systems, anti-CD40 treatment alone can result in ineffective inflammatory cytokine production.(48), the deletion of antigen-specific T cells (Mauri et al. Nat Med 6:673 (2001); Kedl et al. Proc Natl Acad Sci., USA 98:10811 (2001)) and termination of B cell responses (Erickson et al., J Clin Invest 109:613 (2002)). Also, soluble trimerized CD40 ligand has been used n the clinic as an agonist for the CD40 pathway and what little has been reported is consistent with the conclusion that stimulation of CD40 alone fails to reconstitute all necessary signals for long term CD8+ T cell immunity (Vonderheide et al., J Clin Oncol 19:3280 (2001)).

Because of the activity of TLRs and CD40 in innate and adaptive immune responses, both of these molecules have been explored as targets for vaccine adjuvants. Recently, it was demonstrated that immunization with antigen in combination with some TLR agonists and anti-CD40 treatment (combined TLR/CD40 agonist immunization) induces potent CD8+ T cell expansion, elicting a response 10-20 fold higher than immunization with either agonist alone (Ahonen et al., J Exp Med 199:775 (2004)). This was the first demonstration that potent CD8+ T cell responses can be generated in the absence of infection with a viral or microbial agent. Antigen specific CD8+ T cells elicited by combined TLR/CD40 agonist immunization demonstrate lytic function, gamma interferon production, and enhanced secondary responses to antigenic challenge. Synergistic activity with anti-CD40 in the induction of CD8+ T cell expansion has been shown with agonists of TLR1/6, 2/6, 3, 4, 5, 7 and 9. This suggests that combined TLR/CD40 agonist immunization can reconstitute all of the signals required to elicit profound acquired cell-mediated immunity.

To increase the effectiveness of an adaptive immune response, such as in a vaccination protocol or during a microbial infection, it is therefore important to develop novel, more effective, vaccine adjuvants. The present invention satisfies this need and provides other advantages as well.

### SUMMARY OF THE INVENTION

This invention relates to synergistic immune adjuvants comprising the combination of (i) at least one live, inactivated or dead whole microorganism or portion thereof (other than a specific isolated compound such a flagellin) which functions as a toll-like receptor (TLR) agonist, i.e. which microorganism or portion thereof on in vivo administration agonizes at least one TLR and (ii) at least one 4-1BB or CD40 agonist such as a CD40 or 4-1BB agonistic antibody or fragment thereof or a monomeric or multimeric (trimeric) CD40L or 4-1BB ligand protein, CD40L protein fragment, or conjugate containing and (iii) optionally an antigen against which a cellular immune response is desirably elicited. The present invention further relates to the use of such combinations a immune adjuvants and for treating conditions wherein T cell immunity is desirably enhanced.

The use of synergistic adjuvants comprising a TLR agonist and a CD40 agonist or 4-1BB agonist and optionally an antigen is disclosed in U.S. Ser. No. 10/748,010 filed on Dec. 30, 2003 which application is incorporated by reference in its entirety herein. This prior application exemplifies a variety of isolated TLR agonist compounds and their use in conjunction with CD40 and 4-1BB agonists and optionally a desired antigen to which a T cell immune response is desirably to be elicited against and the use thereof as immune adjuvants for treating conditions such as cancer, infection, autoimmune diseases and other conditions wherein antigen specific T cell immunity is desired.

This invention is an extension thereof as it relates to a TLR agonist/CD40 or TLR agonist/4-1BB agonist combination wherein the TLR agonist is an endogenous microbial TLR agonist such as a whole bacterium, yeast, fungi or virus which may be mutated or genetically engineered to express or not express a desired polypeptide, e.g., an antigen or toxin, or may comprise a portion thereof other than an isolated compound, e.g., a membrane thereof, microbial extract, or a spheroplast, cytoplast, or ghost. In a preferred embodiment the microorganism or portion thereof that functions a TLR agonist will comprise a yeast , bacterium or virus such as a recombinant virus, whole bacterium or yeast, yeast or bacterial cytoplast, yeast or bacterial spheroplast, yeast or bacterial ghost, or subcellular yeast particle. In some instances the microbial material such as a whole microorganism or virus may function both as TLR agonist and as an antigen delivery system. For example the virus or portion thereof which functions as a TLR agonist may express an antigen against which T cell immunity is desired such as a viral antigen or a non-viral antigen. Alternatively, a yeast or bacterium or portion thereof which functions as a TLR agonist may endogenously express an antigen to which immunity is desirably elicited or may be loaded with or genetically engineered to express a desired antigen or a CD40 or 4-1BB agonist , e.g., as a fusion protein on the bacterial or yeast surface. Such microbial TLR agonist/CD40 or 4-1BB agonist combination may be administered to a host in need thereof, in order to elicit a synergistic effect on cellular immunity, particularly primary and memory CD8+ T cell responses.

In particular this invention encompasses as the TLR microbial adjuvants for use in combination with a CD40 agonist the yeast immunogenic vehicles which are disclosed in U.S. Pat. No. 7,083,787; U.S. Pat. No. 5,413,914 and U.S. Pat. No. 5,830,463. The contents of these patents is incorporated by reference in their entireties herein.

As described in detail infra, these immune combinations may be administered to a host in need of such treatment as a means of:

(i) generating enhanced (exponentially better) primary and memory CD8+ T cell responses relative to immunization with either agonist alone;

(ii) inducing the exponential expansion of antigen-specific CD8+ T cells, and

(iii) generating protective immunity even in CD4 deficient or depleted hosts.

These immune adjuvant combinations which optionally may further include an antigen may be used in treating any disease or condition wherein the above-identified enhanced cellular immune responses are therapeutically desirable, especially infectious diseases, proliferative disorders such as cancer, allergy, autoimmune disorders, inflammatory disorders, and other chronic diseases wherein enhanced cellular immunity is a desired therapeutic outcome. Preferred applications of the invention include especially the treatment of infectious disorders such as HIV infection and cancer.

### DETAILED DESCRIPTION OF THE INVENTION

The present invention provides a novel synergistic agonistic combination comprising (i) a whole microorganism or constituent thereof other than an isolated compound (e.g. a membrane extract, spheroplast, cytoplast, or ghost) that functions as a TLR agonist and a CD40 agonist (for example a CD40L protein or fragment or derivative or multimeric therof or an agonistic antibody or antibody fragment that binds CD40 preferably human CD40) or a 4-1BB agonist such as a 4-1BB ligand polypeptide or fragment or conjugate or an anti-4-1BB agonistic antibody and optionally an antigen. These adjuvant combinations when administered to a host, preferably a human, may be used to generate enhanced antigen specific cellular immune responses.

In preferred embodiments the TLR agonist will comprise a whole virus or microorganism which may be engineered to express a desired antigen. In some embodiments the microorganism or virus which functions as a TLR agonist may be genetically engineered to express a CD40 agonist or 4-1BB agonist and/or a desired antigen thereby providing the CD40 or 4-1BB agonist, TLR agonist and optional antigen in a single microbial or viral vehicle thereby facilitating administration to a host having a condition wherein enhanced antigen specific cellular immune response are desirably elicited.

Also, the invention provides methods of using said synergistic adjuvant combinations and vehicles containing to a host in which an antigen specific immune response is desirably elicited, for example a person with a chronic disease such as cancer or an infectious or allergic disorder producing said composition.

Still further the invention provides compositions comprising said novel synergistic TLR/CD40 or TLR/4-1BB agonist combinations which are suitable for administration to a host in order to elicit an enhanced antigen-specific cellular immune response.

Particularly, the invention provides novel methods of immunotherapy comprising the administration of said novel synergistic adjuvant combination to a host in need of such treatment in order to elicit an enhanced antigen specific cellular immune response. In preferred embodiments these compositions and conjugates will be administered to a subject with or at risk of developing a cancer, an infection, particularly a chronic infectious diseases e.g., involving a virus, bacteria or parasite; or an autoimmune, inflammatory or allergic condition. In an exemplary and preferred embodiment , the invention may be used to elicit antigen specific cellular immune responses against HIV. HIV is a well recognized example of a disease wherein protective immunity almost certainly will require the generation of potent and long-lived cellular immune responses against the virus.

While it has been previously reported that TLR agonists synergize with anti-CD40 for the induction of CD8+ T cell immunity. to date all these studies have used as the TLR agonist a discrete compound or have required the separate administration of the antigen, and the CD40 agonist. By contrast this invention provides adjuvant combinations wherein the TLR agonist is a whole microorganism or virus or portion thereof which may optionally may be recombinant and express a desired antigen thereby permitting the antigen and the TLR agonist to comprise the same administered entity. Also, in some embodiments of the invention the microorganism or virus may be genetically engineered to express the CD40 or 4-1BB agonist, e.g., an anti-CD40 or anti-4-1BB agonistic antibody such as a scFv or an intact immunoglobulin or antibody fragment or a CD40L or 4-1BB ligand fusion protein or fragment or variant thereof. This will simplify the use thereof for vaccine or therapeutic purposes since only one entity will need to be formulated in pharmaceutically acceptable form and administered. This is particularly advantageous in the context of treatment of a chronic diseases or conditions wherein large amounts of adjuvant may be required for effective prophylactic or therapeutic immunity.

Thus, this invention provides for the development of potent vaccines against HIV and other chronic infectious diseases involving viruses, bacteria, fungi or parasites as well as proliferative diseases such as cancer, autoimmune diseases, allergic disorders, and inflammatory diseases where effective treatment requires the quantity and quality of cellular immunity that combined TLR/CD40 agonist immunization is capable of generating.

In some embodiments the microbial TLR agonist may also be engineered to express a type I interferon such as alpha interferon or beta interferon or an interferon inducer or CD70 agonist or be co-administered therewith.

### APPLICATIONS OF THE INVENTION

The invention provides novel adjuvant combinations comprising at least one microorganism or extract thereof such as a membrane extract, spheroplast, cytoplast, at least one CD40 or 4-1BB agonist and optionally an antigen wherein the antigen and the CD40 agonist or 4-1BB agonist may be discrete or comprised in the TLR microbial material such as a recombinant yeast or bacterium or virus that expresses the CD40 or 4-1BB agonist and/or a desired antigen. A toll-like receptor agonist herein is intended to encompass any live or dead microorganism or virus or portion or extract thereof other than a discrete isolated compound that elicits a TLR agonist response upon administration to a host . This includes in particular non-pathogenic bacteria, viruses and yeast such as Saccharomyces, Pichia, Hansenula, Cryptococcus, Candida, Hansenula, Kluyveromyces, Rhodotorula, Schizzosaccharomyces, and Yarrowia and membranes, spheroplasts, cytoplasts, ghosts, and subcellular particles derived therefrom. As mentioned, these microbia may express an HIV antigen such as HIVGag40 because HIV is a chronic infectious disease wherein an enhanced cellular immune response has significant therapeutic potential. However, the invention embraces the use of any antigen in combination with the subject microbial derived TLR agonists and CD40 agonists against which an enhanced cellular immune response is therapeutically desirable. In the preferred embodiment the antigen is comprised in the administered microorganism or virus. In some embodiments the antigen may be administered separate from the microbial TLR agonist, or the host may be naturally exposed to the antigen. Additionally, in some embodiments all three moieties, i.e., the CD40 agonist such as anti-CD40 antibody, the microbial TLR agonist and the antigen may be co-administered as separate discrete entities. Preferably all these moieties are administered substantially concurrently in order to achieve the desired synergistic enhancement in cellular immunity. These moieties may be administered in any order.

Exemplary antigens include but are not limited to bacterial, viral, parasitic, allergens, autoantigens and tumor associated antigens. Particularly, the antigen can include protein antigens, peptides, whole inactivated organisms, and the like.

Specific examples of antigens that can be used in the invention include antigens from hepatits A, B, C or D, influenza virus, Listeria, Clostridium botulinum, tuberculosis, tularemia, Variola major (smallpox), viral hemorrhagic fevers, Yersinia pestis (plague), HIV, herpes, pappilloma virus, and other antigens associated with infectious agents. Other antigens include antigens associated with a tumor cell, antigens associated with autoimmune conditions, allergy and asthma. Administration of such an antigen in conjunction with the subject agonist combination can be used in a therapeutic or prophylactic vaccine for conferring immunity against such disease conditions.

In some embodiments the methods and compositions can be used to treat an individual at risk of having an infection or has an infection by including an antigen from the infectious agent. An infection refers to a disease or condition attributable to the presence in the host of a foreign organism or an agent which reproduce within the host. A subject at risk of having an infection is a subject that is predisposed to develop an infection. Such an individual can include for example a subject with a known or suspected exposure to an infectious organism or agent. A subject at risk of having an infection can also include a subject with a condition associated with impaired ability to mount an immune response to an infectious agent or organism, for example a subject with a congenital or acquired immunodeficiency, a subject undergoing radiation or chemotherapy, a subject with a burn injury, a subject with a traumatic injury, a subject undergoing surgery, or other invasive medical or dental procedure, or similarly immunocompromised individual.

Infections which may be treated or prevented with the vaccine compositions of this invention include bacterial, viral, fungal, and parasitic. Other less common types of infection also include are rickettsiae, mycoplasms, and agents causing scrapie, bovine spongiform encephalopathy (BSE), and prion diseases (for example kuru and Creutzfeldt-Jacob disease). Examples of bacteria, viruses, fungi, and parasites that infect humans are well know. An infection may be acute, subacute, chronic or latent and it may be localized or systemic. Furthermore, the infection can be predominantly intracellular or extracellular during at least one phase of the infectious organism's agent's life cycle in the host.

Bacteria infections against which the subject vaccines and methods may be used include both Gram negative and Gram positive bacteria. Examples of Gram positive bacteria include but are not limited to Pasteurella species, Staphylococci species, and Streptococci species. Examples of Gram negative bacteria include but are not limited to Escherichia coli, Pseudomonas species, and Salmonella species. Specific examples of infectious bacteria include but are not limited to Heliobacter pyloris, Borrelia burgdorferi, Legionella pneumophilia, Mycobacteria spp. (for example M. tuberculosis, M. avium, M. intracellilare, M. kansaii, M. gordonae), Staphylococcus aureus, Neisseria gonorrhoeae, Neisseria meningitidis, Listeria monocytogeners, Streptococcus pyogenes, (group A Streptococcus), Streptococcus agalactiae (Group B Streptococcus), Streptococcus (viridans group), Streptococcus faecalis, streptococcus bovis, Streptococcus (aenorobic spp.), Streptococcus pneumoniae, pathogenic Campylobacter spp., Enterococcus spp., Haemophilus influenzae, Bacillus anthracis, Corynebacterium diptheriae, Corynebacterium spp., Erysipelothrix rhusiopathie, Clostridium perfringens, Clostridium tetani, Enterobacter aerogenes, Klebsiella pneumoniae, Pasteurella multocida, Bacteroides spp., Fusobacterium nucleatum, Streptobacillus moniliformis, Treponema pallidum, Treponema pertenue, Leptospira, Rickettsia, and Actinomyces israelii.

Examples of viruses that cause infections in humans include but are not limited to Retroviridae (for example human deficiency viruses, such as HIV-1 (also referred to as HTLV-III), HIV-II, LAC or IDLV-III/LAV or HIV-III and other isolates such as HIV-LP, Picornaviridae (for example poliovirus, hepatitis A, enteroviruses, human Coxsackie viruses, rhinoviruses, echoviruses), Calciviridae (for example strains that cause gastroenteritis), Togaviridae (for example equine encephalitis viruses, rubella viruses), Flaviviridae (for example dengue viruses, encephalitis viruses, yellow fever viruses) Coronaviridae (for example coronaviruses), Rhabdoviridae (for example vesicular stomata viruses, rabies viruses), Filoviridae (for example Ebola viruses) Paramyxoviridae (for example parainfluenza viruses, mumps viruses, measles virus, respiratory syncytial virus), Orthomyxoviridae (for example influenza viruses), Bungaviridae (for example Hataan viruses, bunga viruses, phleoboviruses, and Nairo viruses), Arena viridae (hemorrhagic fever viruses), Reoviridae (for example reoviruses, orbiviruses, rotaviruses), Bimaviridae, Hepadnaviridae (hepatitis B virus), Parvoviridae (parvoviruses), Papovaviridae (papilloma viruses, polyoma viruses), Adenoviridae (adenoviruses), Herpeviridae (for example herpes simplex virus (HSV) I and II, varicella zoster virus, pox viruses) and Iridoviridae (for example African swine fever virus) and unclassified viruses(for example the etiologic agents of Spongiform encephalopathies, the agent of delta hepatitis, the agents of non-A, non-B hepatitis (class 1 enterally transmitted; class 2 parenterally transmitted such as Hepatitis C); Norwalk and related viruses and astroviruses).

Examples of fungi include Aspergillus spp., Coccidoides immitis, Cryptococcus neoformans, Candida albicans and other Candida spp., Blastomyces dermatidis, Histoplasma capsulatum, Chlamydia trachomatis, Nocardia spp., and Pneumocytis carinii.

Parasites include but are not limited to blood-borne and/or tissue parasites such as Babesia microti, Babesi divergans, Entomoeba histolytica, Giarda lamblia, Leishmania tropica, Leishmania spp., Leishmania braziliensis, Leishmania donovdni, Plasmodium falciparum, Plasmodium malariae, Plasmodium ovale, Plasmodium vivax, Toxoplasma gondii, Trypanosoma gambiense and Trypanosoma rhodesiense (African sleeping sickness), Trypanosoma cruzi (Chagus' disease) and Toxoplasma gondii, flat worms, and round worms.

As noted this invention further embraces the use of the subject conjugates in treating proliferative diseases such as cancers. Cancer is a condition of uncontrolled growth of cells which interferes with the normal functioning of bodily organs and systems. A subject that has a cancer is a subject having objectively measurable cancer cells present in the subjects' body. A subject at risk of developing cancer is a subject predisposed to develop a cancer, for example based on family history, genetic predisposition, subject exposed to radiation or other cancer-causing agent. Cancers which migrate from their original location and seed vital organs can eventually lead to the death of the subject through the functional deterioration of the affected organ. Hematopoietic cancers, such as leukemia, are able to out-compete the normal hematopoietic compartments in a subject thereby leading to hematopoietic failure (in the form of anemia, thrombocytopenia and neutropenia), ultimately causing death.

A metastasis is a region of cancer cells, distinct from the primary tumor location, resulting from the dissemination of cancer cells from the primary tumor to other parts of the body. At the time of diagnosis of the primary tumor mass ,the subject may be monitored for the presence of metastases. Metastases are often detected through the sole or combined use of magnetic resonance imaging (MRI), computed tomography (CT), scans, blood and platelet counts, liver function studies, chest -X-rays and bone scans in addition to the monitoring of specific symptoms.

The adjuvant combinations and compositions containing according to the invention can be used to treat a variety of cancers or subjects at risk of developing cancer, by the inclusion of a tumor-associated-antigen (TAA), or DNA encoding. This is an antigen expressed in a tumor cell. Examples of such cancers include breast, prostate, colon, blood cancers such as leukemia, chronic lymphocytic leukemia, and the like. The vaccination methods of the invention can be used to stimulate an immune response to treat a tumor by inhibiting or slowing the growth of the tumor or decreasing the size of the tumor. A tumor associated antigen can also be an antigen expressed predominantly by tumor cells but not exclusively.

Additional cancers include but are not limited to basal cell carcinoma, biliary tract cancer, bladder cancer, bone cancer, brain and central nervous system (CNS) cancer, cervical cancer, choriocarcinoma, colorectal cancers, connective tissue cancer, cancer of the digestive system, endometrial cancer, esophageal cancer, eye cancer, head and neck cancer, gastric cancer, intraepithelial neoplasm, kidney cancer, larynx cancer, liver cancer, lung cancer (small cell, large cell), lymphoma including Hodgkin's lymphoma and non-Hodgkin's lymphoma; melanoma; neuroblastoma; oral cavity cancer(for example lip, tongue, mouth and pharynx); ovarian cancer; pancreatic cancer; retinoblastoma; rhabdomyosarcoma; rectal cancer; cancer of the respiratory system; sarcoma; skin cancer; stomach cancer; testicular cancer; thyroid cancer; uterine cancer; cancer of the urinary system; as well as other carcinomas and sarcomas.

The adjuvant combinations and compositions containing according to the invention can also be used to treat autoimmune diseases such as multiple sclerosis, rheumatoid arthritis, type 1 diabetes, psoriasis or other autoimmune disorders. Other autoimmune disease which potentially may be treated with the vaccines and immune adjuvants of the invention include Crohn's disease and other inflammatory bowel diseases such as ulcerative colitis, systemic lupus eythematosus (SLE), autoimmune encephalomyelitis, myasthenia gravis (MG), Hashimoto's thyroiditis, Goodpasture's syndrome, pemphigus, Graves disease, autoimmune hemolytic anemia, autoimmune thrombocytopenic purpura, scleroderma with anti-collagen antibodies, mixed connective tissue disease, polypyositis, pernicious anemia, idiopathic Addison's disease, autoimmune associated infertility, glomerulonephritis)for example crescentic glomerulonephritis, proliferative glomerulonephritis), bullous pemphigoid, Sjogren's syndrome, psoriatic arthritis, insulin resistance, autoimmune diabetes mellitus (type 1 diabetes mellitus; insulin dependent diabetes mellitus), autoimmune hepatitis, autoimmune hemophilia, autoimmune lymphoproliferative syndrome (ALPS), autoimmune hepatitis, autoimmune hemophilia, autoimmune lymphoproliferative syndrome, autoimmune uveoretinitis, and Guillain-Bare syndrome. Recently, arteriosclerosis and Alzheimer's disease have been recognized as autoimmune diseases. Thus, in this embodiment of the invention the antigen will be a self-antigen against which the host elicits an unwanted immune response that contributes to tissue destruction and the damage of normal tissues.

The adjuvant combinations and compositions containing according to the invention can also be used to treat asthma and allergic and inflammatory diseases. Asthma is a disorder of the respiratory system characterized by inflammation and narrowing of the airways and increased reactivity of the airways to inhaled agents. Asthma is frequently although not exclusively associated with atopic or allergic symptoms. Allergy is acquired hypersensitivity to a substance (allergen). Allergic conditions include eczema, allergic rhinitis, or coryza, hay fever, bronchial asthma, urticaria, and food allergies and other atopic conditions. An allergen is a substance that can induce an allergic or asthmatic response in a susceptible subject. There are numerous allergens including pollens, insect venoms, animal dander, dust, fungal spores, and drugs.

Examples of natural and plant allergens include proteins specific to the following genera: Canine, Dermatophagoides, Felis, Ambrosia, Lotium, Cryptomeria, Alternaria, Alder, Alinus, Betula, Quercus, Olea, Artemisia, Plantago, Parietaria, Blatella, Apis, Cupressus, Juniperus, Thuya, Chamaecyparis, Periplanet, Agopyron, Secale, Triticum, Dactylis, Festuca, Poa, Avena, Holcus, Anthoxanthum, Arrhenatherum, Agrostis, Phleum, Phalaris, Paspalum, Sorghum, and Bromis.

It is understood that the adjuvant combinations and compositions containing according to the invention can be combined with other therapies for treating the specific condition, e.g., infectious disease, cancer or autoimmune condition. For example in the case of cancer the inventive methods may be combined with chemotherapy or radiotherapy.

Methods of making compositions as vaccines are well known to those skilled in the art. The effective amounts of the microbial TLR agonist, CD40 or 4-1BB agonist and antigen can be determined empirically, but can be based on immunologically effective amounts in animal models. Factors to be considered include the antigenicity, the formulation, the route of administration, the number of immunizing doses to be administered, the physical condition, weight, and age of the individual, and the like. Such factors are well known to those skilled in the art and can be determined by those skilled in the art (see for example Paoletti and Mclnnes,eds., Vaccines, from Concept to Clinic: A Guide to the Development and Clinical Testing of Vaccines for Human Use CRC Press (1999). As disclosed herein it is understood that the subject DNAs or protein conjugates can be administered alone or in conjunction with other adjuvants.

The adjuvants of the invention can be administered locally or systemically by any method known in the art including but not limited to intramuscular, intravenous, intradermal, subcutaneous, intraperitoneal, intranasal, oral or other mucosal routes. Additional routes include intracranial (for example intracisternal, or intraventricular), intraorbital, ophthalmic, intracapsular, intraspinal, and topical administration. The adjuvants and vaccine compositions of the invention can be administered in a suitable, nontoxic pharmaceutical carrier, or can be formulated in microcapsules or a sustained release implant. The immunogenic compositions of the invention can be administered multiple times, if desired, in order o sustain the desired cellular immune response. The appropriate route, formulation, and immunization schedule can be determined by one skilled in the art.

In the methods of the invention, in some instances the antigen and a microbial TLR/CD40 agonist conjugate may be administered separately or combined in the same formulation. In some instances it may be useful to include several antigens. These compositions may be administered separately or in combination in any order that achieve the desired synergistic enhancement of cellular immunity. Typically, these compositions are administered within a short time of one another, i.e. within about several hours of one another, more preferably within about a half hour. In some embodiments they may be co-administered within about 24-48 hours of one another.

In some instances, it may be beneficial to include a moiety in the adjuvant which facilitates affinity purification. Such moieties include relatively small molecules that do not interfere with the function of the adjuvant combination. Alternatively, the tags may be removable by cleavage. Examples of such tags include poly-histidine tags, hemagglutinin tags, maltase binding protein, lectins, glutathione-S transferase, avidin and the like. Other suitable affinity tags include FLAG, green fluorescent protein (GFP), myc, and the like.

The subject adjuvant combinations can be administered with a physiologically acceptable carrier such as physiological saline. The composition may also include another carrier or excipient such as buffers, such as citrate, phosphate, acetate, and bicarbonate, amino acids, urea, alcohols, ascorbic acid, phospholipids, proteins such as serum albumin, ethylenediamine tetraacetic acid, sodium chloride or other salts, liposomes, mannitol, sorbitol, glycerol and the like. The adjuvants of the invention can be formulated in various ways, according to the corresponding route of administration. For example, liquid formulations can be made for ingestion or injection, gels or procedures can be made for ingestion, inhalation, or topical application. Methods for making such formulations are well known and can be found in for example, “Remington's Pharmaceutical Sciences,” 18^{th} Ed., Mack Publishing Company, Easton Pa.

The invention also embraces DNA based vaccines. These DNAs which may encode a desired antigen and/or CD40 adjuvant may be administered as naked DNAs, or may be comprised in an expression vector such as a recombinant virus that functions as the TLR agonist. Furthermore, the subject nucleic acid sequences may be introduced into a cell of a graft prior to transplantation of the graft. This DNA preferably will be humanized to facilitate expression in a human subject.

The subject adjuvant combinations may further include a “marker” or “reporter”. Examples of marker or reporter molecules include beta lactamase, chloramphenicol acetyltransferase, adenosine deaminase, aminoglycoside phosphotransferase, dihydrofolate reductase, hygromycin B-phosphotransferase, thymidine kinase, lacZ, and xanthine guanine phosphoribosyltransferase et al.

The subject microbial TLR adjuvants can contain a vector capable of directing the expression of an antigen or CD40 or 4-1BB agonist, for example a cell transduced with the vector. For example a baculovirus vector can be used. Other vectors which may be used include T7 based vectors for use in bacteria, yeast expression vectors, mammalian expression vectors, viral expression vectors, and the like. Viral vectors include retroviral, adenoviral, adeno-associated vectors, herpes virus, simian virus 40, and bovine papilloma virus vectors. Also, bacterial and yeast expression vectors are preferably used in conjunction with a yeast or bacterial TLR agonist

Prokaryotic and eukaryotic cells that may function as TLR agonists or which can be used to facilitate expression of the subject adjuvants or antigens include by way of example microbia, plant and animal cells, e.g., prokaryotes such as Escherichia coli, Bacillus subtilis, and the like, insect cells such as Sf21 cells, yeast cells such as Saccharomyces, Candida, Kluyveromyces, Schizzosaccharomyces, and Pichia, and mammalian cells such as COS, HEK293, CHO, BHK, NIH 3T3, HeLa, and the like. One skilled in the art can readily select appropriate components for a particular expression system, including expression vector, promoters, selectable markers, and the like suitable for a desired cell or organism. The selection and use of various expression systems can be found for example in Ausubel et al., “Current Protocols in Molecular Biology, John Wiley and Sons, New York, N.Y. (1993); and Pouwels et al., Cloning Vectors: A Laboratory Manual”:, 1985 Suppl. 1987). Also provided are eukaryotic cells that contain and express the subject DNA constructs.

In the case of cell transplants, the cells can be administered either by an implantation procedure or with a catheter-mediated injection procedure through the blood vessel wall. In some cases, the cells may be administered by release into the vasculature, from which the cells subsequently are distributed by the blood stream and/or migrate into the surrounding tissue.

The CD40 agonists or 4-1BB agonists as noted preferably comprise an agonistic anti-CD40 antibody or anti-4-1BB agonistic antibody or fragment thereof that specifically binds CD40 or 4-1BB , preferably murine or human CD40 or human 4-1BB or a CD40L or 4-1BB ligand protein, derivative, multimer such as a trimeric CD40L or 4-1BB ligand conjugate. As used herein, the term “antibody” is used in its broadest sense to include polyclonal and monoclonal antibodies, as well as antigen binding fragments thereof. This includes Fab, F(ab′)2, Fd and Fv fragments.

In addition the term “antibody” includes naturally antibodies as well as non-naturally occurring antibodies such as single chain antibodies, chimeric antibodies, bifunctional and humanized antibodies. Preferred for use in the invention are chimeric, humanized and fully human antibodies. Methods for synthesis of chimeric, humanized, CDR-grafted, single chain and bifunctional antibodies are well known to those skilled in the art. In addition, antibodies specific to CD40 or 4-1BB antigen are widely known and available and can be made by immunization of a suitable host with a CD40 antigen, preferably human CD40.

It is understood that modifications which do not substantially affect the activity of the various embodiments of this invention are also provided within the definition of the invention provided herein.

The various references to journals, patents, and other publications which are cited herein comprise the state of the art and are incorporated by reference as though fully set forth.

1-38. (canceled)

39. An adjuvant combination which elicits a synergistic effect on T cell immunity comprising a recombinant microorganism or virus that expresses on its surface all of the following: (i) at least one agonist of CD40 or 4-1BB; (ii) at least one microbial TLR agonist other than a flagellin polypeptide; (iii) a type 1 interferon and (iv) optionally at least one desired antigen.

40. The adjuvant combination of claim 39 comprising a recombinant virus.

41. The adjuvant combination of claim 39 wherein the microorganism is a yeast or bacterium or fungi.

42. The adjuvant combination of claim 39 wherein the TLR agonist is an agonist of a TLR selected from TLR1, TLR2, TLR3, TLR4, TLR5, TLR6, TLR7, TLR8, TLR9, TLR10, TLR11, and TLR12.

43. The adjuvant combination of claim 41 wherein the yeast is a Saccharomyces, Candida, Pichia, Rhodotorula, Schizzosaccharomyces, Cryptococcus, Hansenula, Kluyveromyces, or Yarrowia or spheroplast, cytoplast, or yeast ghost derived therefrom.

44. The adjuvant combination of claim 39 wherein the CD40 agonist is an anti-CD40 antibody or antibody fragment or a CD40L protein, derivative, fragment, or multimer thereof or a conjugate containing and the 4-1BB agonist is selected from an agonistic anti-4-1BB antibody or antibody fragment or a 4-1BB ligand protein, derivative, fragment, multimer or conjugate thereof.

45. The adjuvant combination of claim 44 wherein said immunoglobulin is a chimeric immunoglobulin.

46. The adjuvant combination of claim 45 wherein said immunoglobulin is a humanized immunoglobulin.

47. The adjuvant combination of claim 45 wherein said immunoglobulin is a human immunoglobulin.

48. The adjuvant combination of claim 45 wherein said immunoglobulin is a single chain immunoglobulin.

49. The adjuvant combination of claim 39 wherein said antigen is a viral, bacterial, fungal, or parasitic antigen.

50. The adjuvant combination of claim 39 wherein said antigen is a human antigen.

51. The adjuvant combination of claim 50 wherein said human antigen is a cancer antigen, autoantigen or other human antigen the expression of which correlates or is involved in a chronic human disease.

52. The adjuvant combination of claim 49 wherein said viral antigen is specific to a virus selected from the group consisting of HIV, herpes, papillomavirus, ebola, picorna, enterovirus, measles virus, mumps virus, bird flu virus, rabies virus, VSV, dengue virus, hepatitis virus, rhinovirus, yellow fever virus, bunga virus, polyoma virus, coronavirus, rubella virus, echovirus, pox virus, varicella zoster, African swine fever virus, influenza virus and parainfluenza virus.

53. The adjuvant combination of claim 49 wherein said bacterial antigen is derived from a bacterium selected from the group consisting of Salmonella, Escherichia, Pseudomonas, Bacillus, Vibrio, Campylobacter, Heliobacter, Erwinia, Borrelia, Pelobacter, Clostridium, Serratia, Xanothomonas, Yersinia, Burkholdia, Listeria, Shigella, Pasteurella, Enterobacter, Corynebacterium and Streptococcus.

54. The adjuvant combination of claim 49 wherein said parasite antigen is derived from a parasite selected from Babesia, Entomoeba, Leishmania, Plasmodium, Trypanosoma, Toxoplasma, Giarda, flat worms and round worms.

55. The adjuvant combination of claim 49 wherein said fungal antigen is derived from a fungi selected from the group consisting of Aspergillus, Coccidoides, Cryptococcus, Candida Nocardia, Pneumocystis, and Chlamydia.

56. The adjuvant combination of claim 39 wherein the antigen is a cancer antigen expressed by a human cancer selected from the group consisting of prostate cancer, pancreatic cancer, brain cancer, lung cancer (small or large cell), bone cancer, stomach cancer, liver cancer, breast cancer, ovarian cancer, testicular cancer, skin cancer, lymphoma, leukemia, colon cancer, thyroid cancer, cervical cancer, head and neck cancer, sarcoma, glial cancer, and gall bladder cancer

57. The adjuvant combination of claim 1 wherein the antigen is an autoantigen the expression of which correlates to an autoimmune disease.

58. A method for eliciting an antigen specific cellular immune response by administering a adjuvant combination according to claim 39 or a composition containing said adjuvant combination.

59. The method of claim 58 wherein said administering results in a least one of the following: (i) enhanced primary and memory CD8+ T cell responses relative to the administration of a DNA encoding only a CD40 agonist or TLR agonist; (ii) induces exponential expansion of antigen specific CD8+ T cells; and (iii) generates a protective immune response in a CD4 deficient host comparable to a normal (non-CD4 deficient) host.
